(19) United States
(12) Patent Application Publication
(10) Pub. No.: US 2025/0044182 A1
(43) Pub. Date: Feb. 6, 2025

(54) MAIZE RUNNERS

(71) Applicant: BRIGHAM YOUNG UNIVERSITY, Provo, UT (US)

(72) Inventors: Douglas Cook, Fountain Green, UT (US); Samuel Archibald McKinnon, Provo, UT (US); Jarom Harris, Spanish Fork, UT (US); Clark O'Dell Miller, Provo, UT (US); Suzanna Gilbert, Ephraim, UT (US); Carter Noh, Provo, UT (US)

(73) Assignee: BRIGHAM YOUNG UNIVERSITY, Provo, UT (US)

(21) Appl. No.: 18/766,425

(22) Filed: Jul. 8, 2024

Related U.S. Application Data
(60) Provisional application No. 63/525,528, filed on Jul. 7, 2023.

(51) Int. Cl. G01M 5/00; G01N 33/00; G01N 33/02

(52) U.S. Cl. CPC G01M 5/0025; G01M 5/0075; G01N 33/0098; G01N 33/025

(57) ABSTRACT

Disclosed is a rigidity measuring device comprising an arm. The arm includes a rail and a base connected to the rail. The rail includes a limit switch, and the base includes a load cell. Also, disclosed is a rigidity measuring device comprising an arm and a processor. The arm includes a rail and a base connected to the rail. The rail includes a limit switch, and the base includes a load cell. The processor electronically connects to the limit switch and the load cell.

### BACKGROUND

This application claims the priority and benefit of U.S. Provisional Patent Application No. 63/525,528 filed on Jul. 7, 2023, which is incorporated by reference in its entirety.

Corn was first domesticated in southern Mexico about 9,000 years ago. The wild ancestor of corn is a grass called teosinte. Indigenous peoples of the Americas developed corn through selective breeding from teosinte. By around 1500 BCE, corn had become a staple crop in Mesoamerica. Corn cultivation spread throughout Mesoamerica, becoming a fundamental part of the diet and culture of civilizations like the Maya and Aztecs. Corn spread northward into what is now the United States and Canada, being cultivated by various Native American tribes. Corn also spread southward into regions of South America, becoming a key crop in Andean civilizations.

Corn was introduced to Europe by Christopher Columbus and other explorers following his voyages to the Americas in the late 15th and early 16th centuries. Corn quickly spread throughout Europe, Africa, and Asia, becoming an important crop due to its adaptability to various climates and soils. The 20th century saw significant advancements in corn production, including the development of hybrid varieties, increased use of fertilizers, and mechanized farming techniques. Because of these significant advancements in corn production, corn is one of the most widely grown crops in the world. It is used for human consumption, animal feed, and as a raw material in various industrial products, including ethanol.

Even with the significant advancements in agricultural technology and techniques thousands of acres of corn are destroyed each year by wind-induced failure of the stem. To assist in the development of corn species with stronger stalks, scientists need to be able to measure the structural stiffness of many corn stalks. Determining the stiffness of corn stalks would help scientists to develop varieties of corn that could withstand the wind's fury. Furthermore, farmers are interested in knowing the stiffness of their corn stalks to determine if their crops will survive windstorms.

Previously, a device was created for measuring stiffness in cornstalks. However, this device was difficult to implement because it requires manual operation and takes 20 seconds to measure the stiffness of one stalk. The objective of this disclosure is to present a method, system, and automated device that measures the flexural stiffness of plants in rapidly.

### SUMMARY OF THE DISCLOSURE

Disclosed herein is a method, system, and device for measuring the rigidity of plants.

### BRIEF DESCRIPTION OF THE DRAWINGS

Non-limiting and non-exhaustive implementations of the disclosure are described with reference to the following figures, wherein like reference numerals refer to like parts throughout the various views unless otherwise specified. The advantages of the disclosure will become better understood with regard to the following description and accompanying drawings where:

FIG. 1 illustrates a perspective view of a maize runner arm with a transparent guard and rail to illustrate internal components.

FIG. 2 illustrates an exploded view of a maize runner arm.

FIG. 3 illustrates a front-end view of a maize runner arm with a transparent rail to illustrate internal components.

FIG. 4 illustrates a back-end view of a maize runner arm without a rail or a guard.

FIG. 5 illustrates a top view of a maize runner arm attached to framing.

FIG. 6 illustrates a perspective view of a maize runner arm attached to framing.

FIG. 7 illustrates a top view of a mounted maize runner arm attached to framing.

FIG. 8 illustrates a subsystem diagram of a maize runner arm.

FIG. 9 illustrates a subsystem analysis of a maize runner arm.

### DETAILED DESCRIPTION

In the following description of the disclosure, reference is made to the accompanying drawings, which form a part hereof, and in which are shown by way of illustration-specific implementations in which the disclosure may be practiced. It is understood that other implementations may be utilized, and structural changes may be made without departing from the scope of the disclosure.

In the following description, for purposes of explanation and not limitation, specific techniques and embodiments are set forth, such as particular techniques and configurations, in order to provide a thorough understanding of the device disclosed herein. While the techniques and embodiments will primarily be described in context with the accompanying drawings, those skilled in the art will further appreciate that the techniques and embodiments may also be practiced in other similar devices.

Reference will now be made in detail to the exemplary embodiments, examples of which are illustrated in the accompanying drawings. Wherever possible, the same reference numbers are used throughout the drawings to refer to the same or like parts. It is further noted that elements disclosed with respect to particular embodiments are not restricted to only those embodiments in which they are described. For example, an element described in reference to one embodiment or figure may be alternatively included in another embodiment or figure regardless of whether or not those elements are shown or described in another embodiment or figure. In other words, elements in the figures may be interchangeable between various embodiments disclosed herein, whether shown or not.

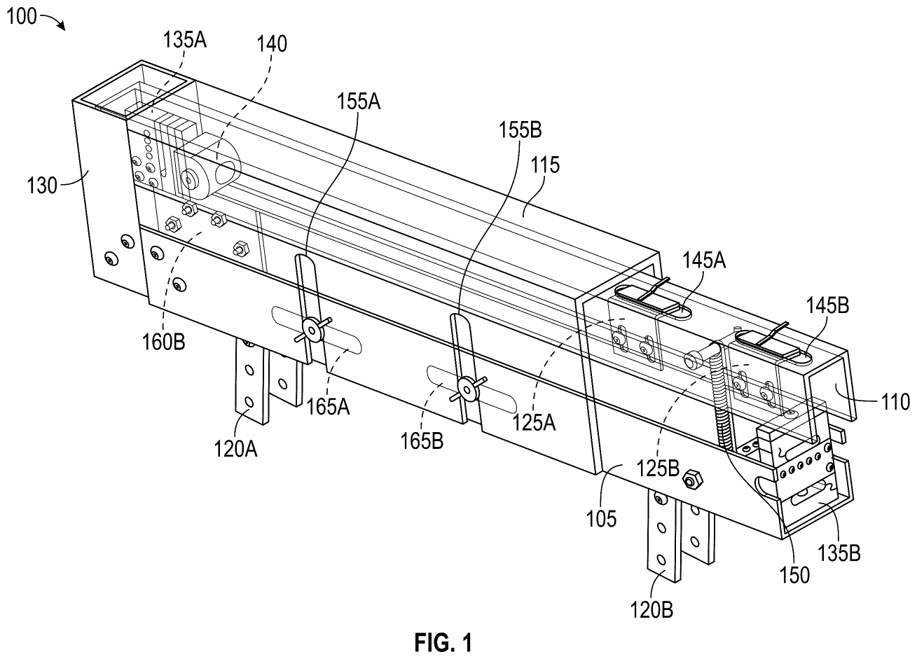

FIG. 1 illustrates a perspective view of maize runner arm 100 with transparent guard 115 and transparent rail 110 to illustrate internal components. Guard 115 may attach to base 105. Base 105 may attach to one or more mounting pivots 120A and 120B. Mounting pivot 120B may be positioned closer to a first end of base 105 while mounting pivot 120A may be positioned closer to a second end of base 105. Near the first end of base 105, one end of spring 145 may attach. The other end of spring 145 may attach to the first end of rail 110. Additionally, load cell 135B may attach at or near to the first end of base 105. Load cell 135B may be positioned closer to the first end of base 105 than the attachment point of spring 145 on base 105. Load cells 135A-B allow arm 100 to measure force. For example, when a corn stalk pushes against or pulls on the rail, the rail transfers this force to a load cell, which takes a force measurement. Other force sensors may be used in place of load cells 135A-B (e.g., force sensitive resistors (“FSRs”) and force transducers). Alternatively, a specialized sensor that measured force and position may be implemented in place of one or more of load cells 135A-B. More specifically the specialized sensor may simultaneously measure the magnitude of an applied force and the position of that force along arm 100.

Arm 100 may also include brace 130 that may attach to a second end of base 105 using one or more attachment plate 160A-B (106A is not seen due to perspective). Brace 130 may extend the length of base 105. Load cell 135A may attach to brace 130. Further, load cell 135A may extend perpendicular to the length of brace 130. Pivot joint 140 may attach to load cell 135A. Load cell 135A may be attached to pivot joint 140 which is connected to rail 110. Near the first end of rail 110 may be resting near load cell 135B and may be held down spring 150 to prevent rail 110 from chattering and sending faulty data.

Rail 110 may attach to pivot joint 140 positioned near the second end of rail 110. Rail 110 may further include slots 145A-B that extend along the length of rail 110. Limit switches 125A-B may attach to rail 110 along the length of rail 110 such that limit switch 125B is closer to the first end of rail 110 than limit switch 125A. Limit switches 125A-B may be positioned to allow their actuators disposed on limit switches 125A-B to extend beyond the rail through slots 145A-B. For example, limit switches 125A-B may be triggered when corn stalks slide over them. This may let the user interpolate displacement. Guard 115 may shield corn stalks from rail 110. Having multiple stalks pushing on rail 110 at a time adds noise to the system. Guard 115 may be a rigid component to prevent that.

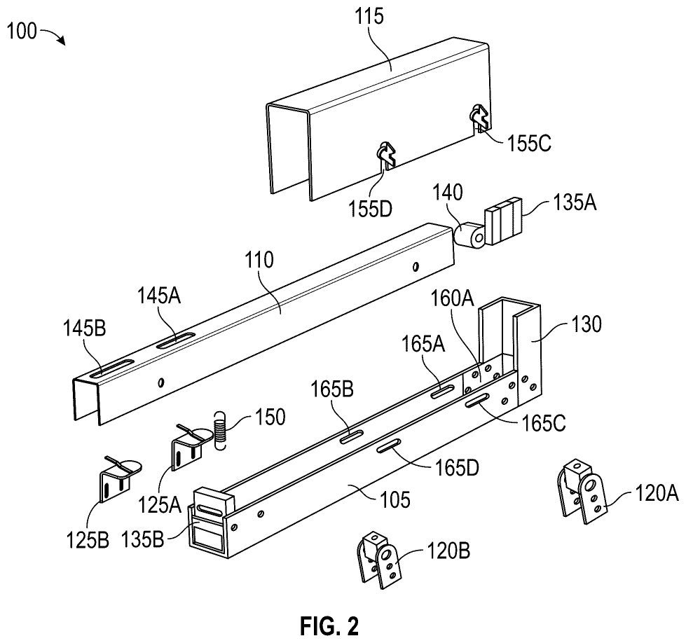

Guard 115 may extend over the top of portions of rail 110 and base 105 while attaching to base 105. Guard 115 may include channels 155A-D that extend perpendicularly to the length of guard 115. Channels 155A-D may open to an outer edge of guard 115 and may be positioned parallel to each other. Channels 155C-D are not seen due to perspective. Channels 155A-D may allow guard 115 to extend further away or be drawn closer to rail 110. Bolts that may include wingnuts may be used to removably attach to base 105. Channels 155A-B may be positioned on one side of guard 115 while channels 155C-D may be positioned on the opposite side of guard 115. Base 105 may also include channels 165A-D that run perpendicular to channels 155A-D which may allow guard 115 to be adjusted towards or away from either end of base 105. Channels 165C-D are not seen due to perspective but are depicted in FIG. 2.

FIG. 2 illustrates an exploded view of a maize runner arm 100. Guard 115 may attach to base 105. Base 105 may attach to one or more mounting pivots 120A and 120B. Mounting pivot 120B may be positioned closer to a first end of base 105 while mounting pivot 120A may be positioned closer to a second end of base 105. Near the first end of base 105, one end of spring 145 may attach. The other end of spring 145 may attach to a first end of rail 110. Additionally, load cell 135B may attach at or near the first end of base 105. Load cell 135 may be positioned closer to the first end of base 105 than the attachment point of spring 145 on base 105. Load cells 135A-B allow arm 100 to measure force. For example, when a corn stalk pushes against or pulls on the rail, the rail transfers this force to a load cell, which takes a force measurement. Other force sensors may be used in place of load cells 135A-B (e.g., force-sensitive resistors (“FSRs”) and force transducers). Alternatively, a specialized sensor that measured force and position may be implemented in place of one or more of load cells 135A-B. More specifically the specialized sensor may simultaneously measure the magnitude of an applied force and the position of that force along arm 100.

Arm 100 may also include brace 130 that may attach to a second end of base 105 using one or more attachment plate 160A-B (106B is not seen due to perspective). Brace 130 may extend perpendicular to the length of base 105. Load cell 135A may attach to brace 130. Further, load cell 135A may extend perpendicular to the length brace 130. Pivot joint 140 may attach to load cell 135. Load cell 135A may be attached to pivot joint 140 which is connected to rail 110. Near the first end of rail 110 may be resting near load cell 135B and may be held down spring 150 as to prevent rail 110 from chattering and sending faulty data.

Rail 110 may attach to pivot joint 140 positioned near the second end of rail 110. Rail 110 may further include slots 145A-B that extend along the length of rail 110. Limit switches 125A-B may attach to rail 110 along the length of rail 110 such that limit switch 125B is closer to the first end of rail 110 than limit switch 125A. Limit switches 125A-B may be positioned to allow their actuators disposed on the limit switches to extend beyond the rail through slots 145A-B. For example, limit switches 125A-B may be triggered when corn stalks slide over them. This may let the user interpolate displacement. Guard 115 may shield corn stalks from rail 110. Having multiple stalks pushing on rail 110 at a time adds noise to the system. Guard may be a rigid component to prevent that.

Guard 115 may extend over top of portions of rail 110 and base 105 while attaching to base 105. Guard 115 may include channels 155A-B that extend perpendicularly to the length of guard 115. Channels 155A-D may open to an outer edge of guard 115 and may be positioned parallel to each other. Channels 155A-B are not seen due to perspective. Channels 155A-D may allow guard 115 to extend further away or be drawn closer to rail 110. Bolts that may include wingnuts may be used to removably attach to base 105. Channels 155A-B may be positioned on one side of guard 115 while channels 155C-D may be positioned on the opposite side of guard 115. Base 105 may also include channels 165A-D that run perpendicular to channels 155A-D which may allow guard 115 to be adjusted towards or away from either end of base 105.

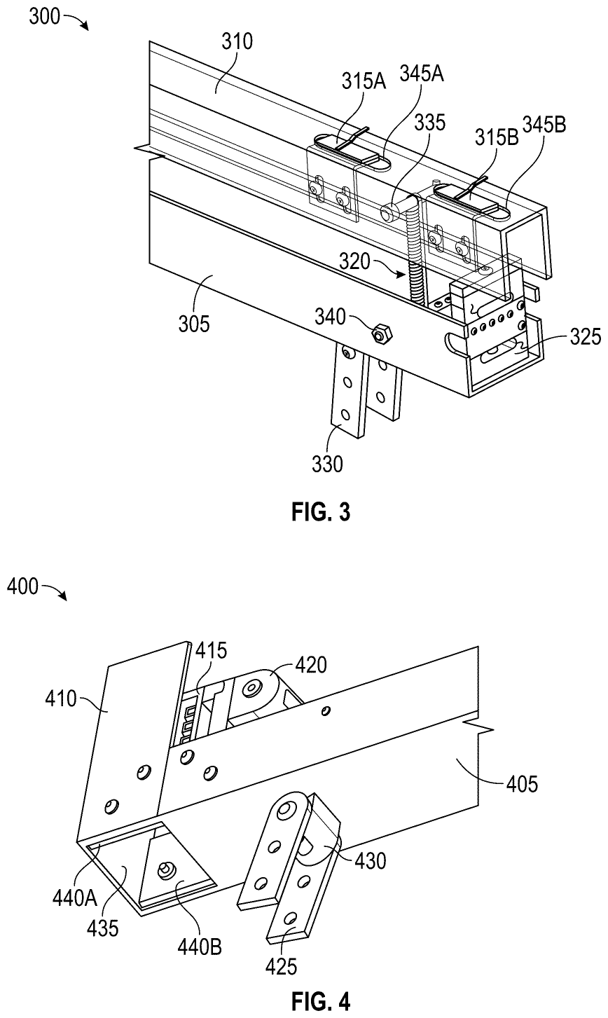

FIG. 3 illustrates a first end view of a maize runner arm 300 with a transparent rail to illustrate internal components. The first end of arm 300 may include base 305 and rail 310. Base 305 may attach to mounting pivot 330. Arm 300 may include spring 320 that attaches to base 305 with connector 340. Connector 340 may include a rod or bolt that extends through base 305. This rod or bolt may include an attachment point to facilitate the attachment of spring 320 to connector 340. Spring 320 is connected to rail 310 with connector 335. Connector 335 may include a rod or bolt that extends through rail 310. This rod or bolt may include an attachment point to facilitate the attachment of spring 320 to connector 335. Limit switches 315A-B may attach to rail 310 along the length of rail 310 such that limit switch 315B is closer to the first end of rail 310 than Limit switch 315A. Limit switches 315A-B may be positioned to allow their actuators disposed on the limit switches 315A-B to extend beyond the rail through slots 345A-B in a linear fashion. Limit switches 315A-B may attach to a single side of rail 310. Additionally, load cell 325 may attach at or near the first end of base 305. Load cell 325 may be closer to the first end of base 305 than where spring 320 attaches to base 305. Further, load cell 325 may be positioned perpendicular to the length of base 305.

FIG. 4 illustrates a second end view of a maize runner arm 400 without a rail or a guard. Arm 400 may include base 405 that may attach to mounting pivot 430. Mounting pivot 430 may attach to mounting bracket 425. Base 405 may also attach to brace 410 and may use attachment plates 440A-B. Brace 410 may extend perpendicular to base 405. Brace 410 may attach load cell 415 and load cell 415 may extend perpendicularly to the length of brace 410. Load cell 415 may attach to pivot 420. Pivot 410 may then attach to the rail as seen in FIG. 1. Arm 400 may also include opening 435 which may allow wires and other cords to connect to internal components positioned within arm 400.

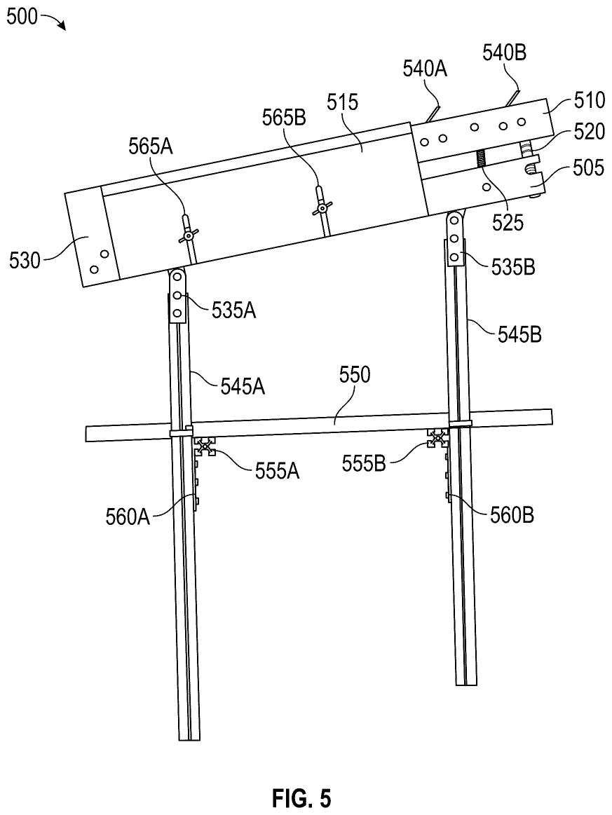

FIG. 5 illustrates a top view of a maize runner arm 500 attached to framing. Arm 500 may include base 505, rail 510, and guard 515. Guard 515 may include channels 565A-D that extend perpendicularly to the length of guard 515. Channels 555A-D may open to an outer edge of guard 515 and may be positioned parallel to each other. Channels 555C-D are not seen due to perspective but are similar to channels 555C-D as shown in FIG. 2. Channels 555A-D may allow guard 515 to extend further away or to be drawn closer to rail 510. Bolts that may include wingnuts may be used to removably attach to base 505. Channels 555A-B may be positioned on one side of guard 515 while channels 555C-D may be positioned on the opposite side of guard 515. Base 505 may also include channels that run perpendicular to channels 555A-D that allow guard 515 to be adjusted towards or away from either end of base 505.

Arm 500 may include spring 525 that may be connected to rail 510 with a connector. Limit switches 540A-B may attach to rail 510 along the length of rail 510 such that limit switch 540B is closer to the first end of rail 510 than limit switch 540A. Further, limit switches 540A-B may attach to rail 510 on a single side and may be positioned in line with one another along a length of rail 510. Limit switches 540A-B may be positioned to allow their actuators disposed on the limit switches 540A-B to extend beyond the rail through slots disposed on rail 510.

Additionally, load cell 520 may attach at or near the first end of base 505. Spring 525 may also attach near the first end of base 505. However, load cell 520 may be closer to the first end of base 505 than where spring 525 may attach to base 505. Base 505 may attach to brace 530 and may be attached using attachment plates (attachment plates are not seen due to perspective however attachment plates in arm 500 are similar to attachment plates 440A-B displayed in FIG. 4). Brace 530 may attach to a load cell (not seen due to perspective but similar to load cell 135A as depicted in FIG. 1). The load cell attached to brace 530 may attach to a pivot. The pivot may attach to rail 510.

Base 505 may also attach to mounting pivots 535A-B. Mounting pivots 535A-B may attach to horizontal framing 545A-B. Mounting pivots 535A-B may allow arm 500 to be set between a 0 and 60 degree angle. Arm 500 may protrude at least 18 inches from the edge of the vehicle. Further, the materials used to make arm 500 may be able to handle more than Horizontal framing may withstand forces greater than 50 pounds. Horizontal framing 545A-B may adjustably attach to vertical framing 555A-B using brackets 560A-B. Vertical framing 555A-B may attach to bar 550. Bar 550 may be positioned perpendicular to horizontal framing 545A-B. Further, bar 550 may be attached to a vehicle (i.e., a truck, a tractor, a robot etc.).

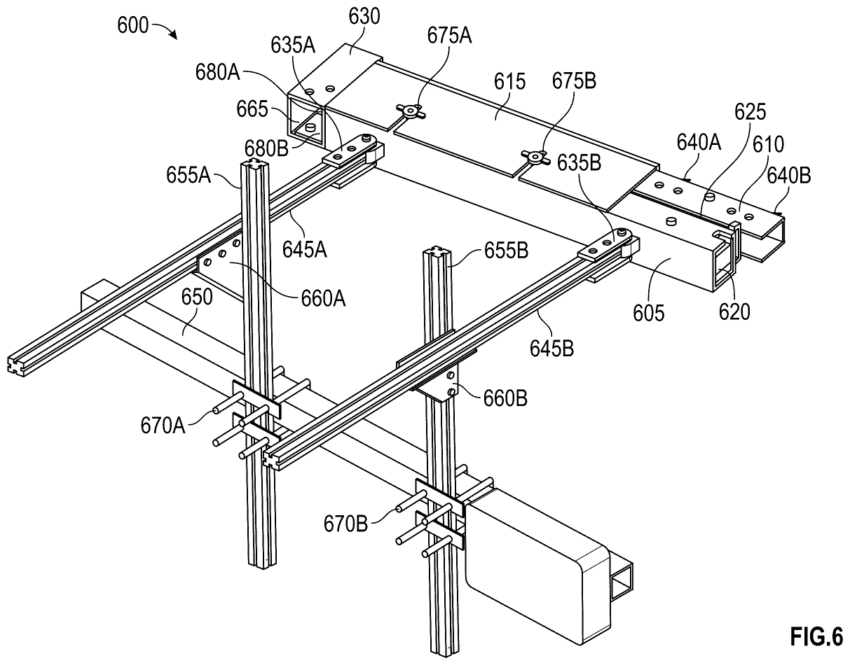

FIG. 6 illustrates a perspective view of a maize runner arm attached to framing. Arm 600 may include base 605, rail 610, and guard 615. Guard 615 may include channels 665A-D that extend perpendicularly to the length of guard 615. Channels 655A-D may open to an outer edge of guard 615 and may be positioned parallel to each other. Channels 655C-D are not seen due to perspective but are similar to channels 655C-D as shown in FIG. 2. Channels 655A-D may allow guard 615 to extend further away or to be drawn closer to rail 610. Bolts that may include wingnuts may be used to removably attach to base 605. Channels 655A-B may be positioned on one side of guard 615 while channels 655C-D may be positioned on the opposite side of guard 615. Base 605 may also include channels that run perpendicular to channels 655A-D that allow guard 615 to be adjusted towards or away from either end of base 605.

Arm 600 may include spring 625 which may be connected to rail 610 with a connector. Limit switches 640A-B may attach to rail 610 along the length of rail 610 such that limit switch 640B is closer to the first end of rail 610 than limit switch 640A. Further, limit switches 640A-B may attach to rail 610 on a single side and may be positioned in line with one another along a length of rail 610. Limit switches 640A-B may be positioned to allow their actuators disposed on the limit switches 640A-B to extend beyond the rail through slots disposed on rail 610.

Additionally, load cell 620 may attach at or near the first end of base 605. Spring 625 may also attach near the first end of base 605. However, load cell 620 may be closer to the first end of base 605 than where spring 625 may attach to base 605. Base 605 may attach to brace 630 and may be attached using attachment plates (attachment plates are not seen due to perspective however attachment plates in arm 600 are similar to attachment plates 440A-B displayed in FIG. 4). Brace 630 may attach to a load cell (not seen due to perspective but similar to load cell 135A as depicted in FIG. 1). The load cell attached to brace 630 may attach to a pivot. The pivot may attach to rail 610.

Base 605 may also attach to mounting pivots 635A-B. Mounting pivots 635A-B may attach to horizontal framing 645A-B. Mounting pivots 635A-B may allow arm 600 to be set between a 0 and 60 degree angle. Arm 600 may protrude at least 18 inches from the edge of the vehicle. Further, the materials used to make arm 600 may be able to handle more than Horizontal framing and may withstand forces greater than 50 pounds. Horizontal framing 645A-B may adjustably attach to vertical framing 655A-B using brackets 660A-B. Vertical framing 655A-B may attach to bar 650. Bar 650 may be positioned perpendicular to horizontal framing 645A-B. Further, bar 650 may be attached to a vehicle (i.e., a truck, a tractor, a robot, etc.).

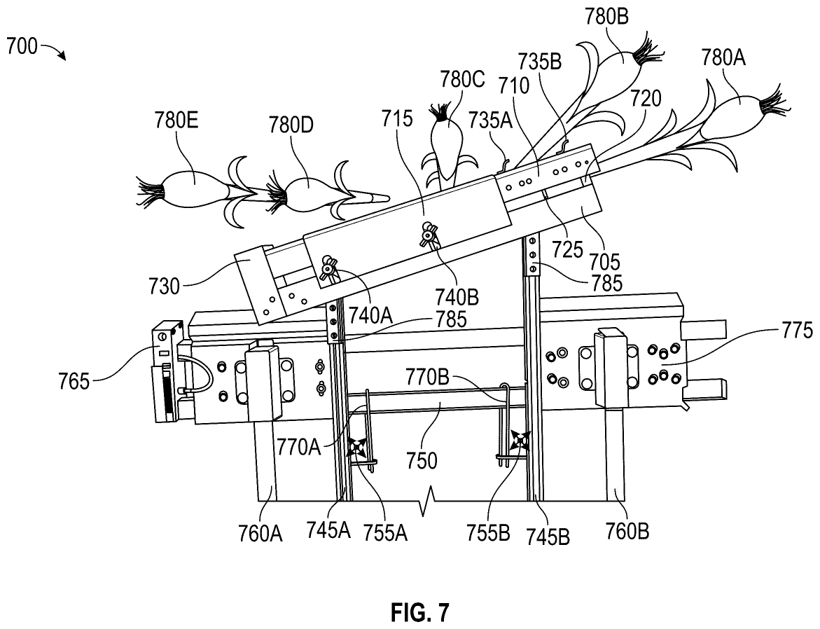

FIG. 7 illustrates a top view of a mounted maize runner arm attached to the framing. Arm 700 may include base 705, rail 710, and guard 715. Guard 615 may include channels 765A-D that extend perpendicularly to the length of guard 715. Channels 755A-D may open to an outer edge of guard 715 and may be positioned parallel to each other. Channels 755C-D are not seen due to perspective but are similar to channels 755C-D as shown in FIG. 2. Channels 755A-D may allow guard 715 to extend further away or to be drawn closer to rail 710. Bolts that may include wingnuts may be used to removably attach to base 705. Channels 755A-B may be positioned on one side of guard 715 while channels 755C-D may be positioned on the opposite side of guard 715. Base 705 may also include channels that run perpendicular to channels 755A-D that allow guard 715 to be adjusted towards or away from either end of base 705.

Arm 700 may include spring 725 that may be connected to rail 710 with a connector. Limit switches 740A-B may attach to rail 710 along the length of rail 710 such that limit switch 740B is closer to the first end of rail 710 than limit switch 740A. Further, limit switches 740A-B may attach to rail 710 on a single side and may be positioned in line with one another along a length of rail 710. Limit switches 740A-B may be positioned to allow their actuators disposed on the limit switches 740A-B to extend beyond the rail through slots disposed on rail 710.

Additionally, load cell 720 may attach at or near the first end of base 705. Spring 725 may also attach near the first end of base 705. However, load cell 720 may be closer to the first end of base 705 than where spring 725 may attach to base 705. Base 705 may attach to brace 730 and may be attached using attachment plates (attachment plates are not seen due to perspective however attachment plates in arm 700 are similar to attachment plates 440A-B displayed in FIG. 4). Brace 730 may attach to a load cell (not seen due to perspective but similar to load cell 135A as depicted in FIG. 1). The load cell attached to brace 730 may attach to a pivot. The pivot may attach to rail 610.

Base 705 may also attach to mounting pivots 735A-B. Mounting pivots 735A-B may attach to horizontal framing 745A-B. Mounting pivots 735A-B may allow arm 700 to be set between a 0 and 60 degree angle. Arm 700 may protrude at least 18 inches from the edge of vehicle 775. Further, the materials used to make arm 700 may be able to handle more than Horizontal framing and may withstand forces greater than 50 pounds. Horizontal framing 745A-B may adjustably attach to vertical framing 755A-B using brackets 760A-B. Vertical framing 755A-B may attach to bar 750. Bar 750 may be positioned perpendicular to horizontal framing 745A-B. Further, bar 750 may be attached to vehicle 775 (i.e., a truck, a tractor, a robot, etc.). Arm 700 may include a global positioning system (“GPS”) and may communicate with vehicle 775 GPS coordinates and may be programed with machine learning technology to navigate through a field and to record the contours of field and compile previous runs to learn which plants are the least rigid in a field. This may provide insight to farmer of poor soil or a pest control issue in a certain area. As machine learning increase arm 700 may be able to map out areas where the plants have similar rigidity and as a result can narrow down the number of plants that need to be tested in a particular field to get an overview of the quality of plants. Further, vehicle 775 may be equipped mapping ability to navigate throughout a field. Arm 700 may also have the ability to include calculations based on temperature, humidity, ground moister and watering schedule to aid in rigidity calculations. As a result, arm 700 may move along a row measuring brushing the arm against the plants. For example, as vehicle 775 moves along a row of maize arm 700 presses against the maize and records and analyzes the data in processor 765 to determine the rigidity of the plants.

For example, arm 700 may receive force measurements from load cell 720 (an additional load sell may be attached to brace 730) and on/off signals from limit switches 735A-B. The stiffness is calculated from these measurement readings recorded on a processor. Arm 700 may move through the cornfield parallel to the rows and may be set at a slight angle to the cornfield. This displaces the cornstalks in a direction perpendicular to the row of corn. Guard 715 may prevent the corn stalks from pressing on rail 710 until a precise location to aid in the measurements. As the corn is displaced, it exerts a force on the arm 700. This force is measured by load cell 720 and recorded by the processor. As the corn slides along the rail 710, it presses the limit switches 735A-B. Detecting where the corn stalks are along arm 700 the processor calculates their displacement perpendicular to the row of corn. Furthermore, this signal lets the user know when the corn stalks are at the edge of rail 710, which lets the processor correlate the force with displacement.

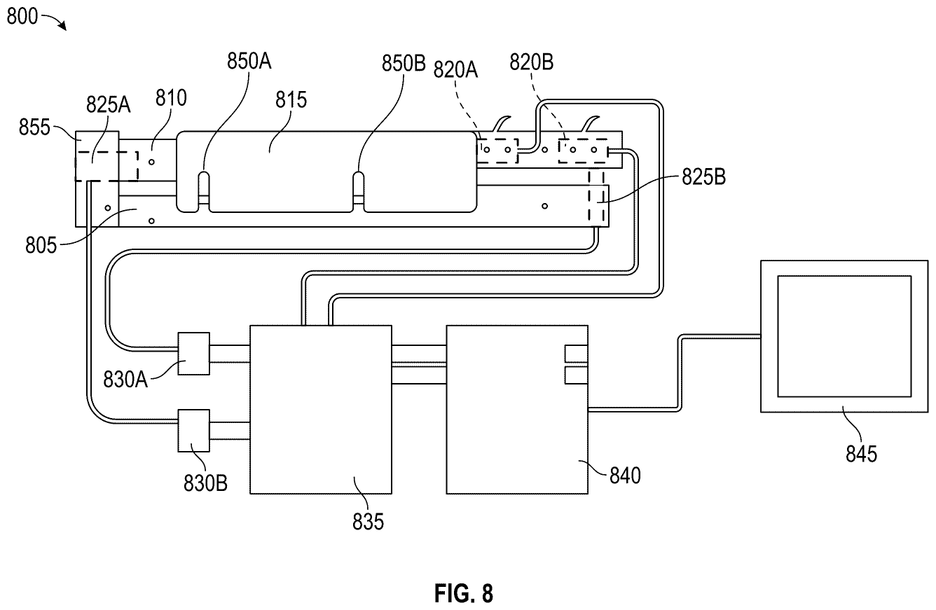

FIG. 8 illustrates a subsystem diagram of a maize runner arm 800. The subsystem of arm 800 includes the basic structure of the measurement devices and devices that perform the analysis. The structure of arm 800 may include base 805, brace 855, rail 810, and guard 815. Base 805 may attach to brace 855 which may connect to rail 810. Guard 815 may include channels 850A-850B this may allow guard 815 to be adjustably attached to base 805. The measurement devices of arm 800 may include load cells 825A-B, limit switches 820A-B, and amplifiers 830A-B. The devices that perform the analysis may include analog to digital converter (“ADC”) 835, processor 840, and display device 845.

The analysis subsystem is made up of the ADC, processor, and the coding involved. The ADC receives the amplified output from the amplifier and the individual signals from the limit switches. The ADC then converts the signals from analog to digital so that the processor can read and compute the data. The software and code on the processor will combine the data from the load cell and limit switches to measure the force vs displacement and then calculate the stiffness of the corn stalk. The ADC and processor will be off the shelf, but the coding will be very customized for this architecture.

For example, arm 800 may use a touch screen interface as display device 845 where the user can select to run tests. Force measurements from load cells 825A-B and on/off signals from limit switches 820A-B may be collected on the processor and exported on a USB drive or other memory device. The stiffness is calculated from these measurements post-processing on the processor. Arm 800 moves through the cornfield parallel to the rows and may be set at a slight angle to the cornfield. This displaces the cornstalks in a direction perpendicular to the row of corn. Guard and rail may be the only part that interferes with the corn stalk and push the stalks as they come and slide across. Guard 815 may prevent the corn stalks from pressing on rail 810 until a precise location to aid in the measurements. As the corn is displaced, it exerts a force on the arm 800. This force is measured by load cells 825A-B and recorded by the processor. As the corn slides along the rail 810, it presses the limit switches 820A-B. Detecting where the corn stalks are along arm 800 the processor calculates their displacement perpendicular to the row of corn. Furthermore, this signal lets the user know when the corn stalks are at the edge of rail 810, which lets the processor correlate the force with displacement.

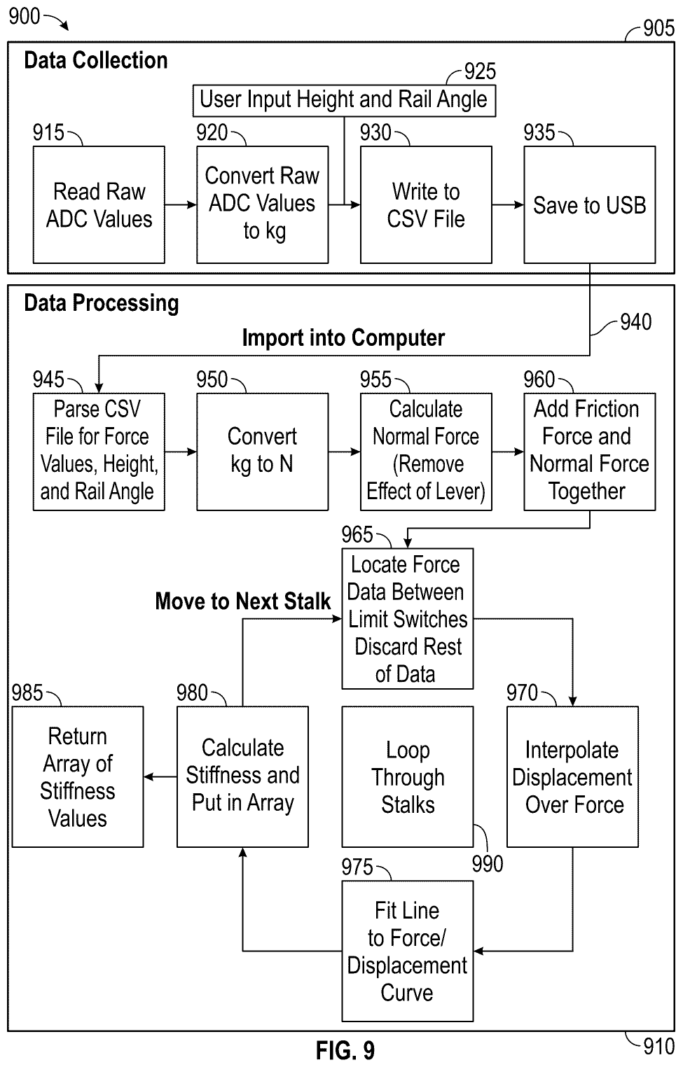

FIG. 9 illustrates a subsystem analysis 900 of a maize runner arm.

Subsystem 900 includes data collection and data processing 905. Step 915 of data collection 905 may be where the processor 840 (as depicted in FIG. 8) reads the raw ADC values 915. Then in step 920, the processor 840 may convert the raw ADC values to kilograms. At step 925 a user may input the height and angle of the rail 810 (as depicted in FIG. 8). In step 930 processor 840 may write a comma-separated value (“CSV”) file 930. The processes may save the information to a storage system 935. Step 935 is data processing 905 which may include storing the information to a USB storage device or other storage device known in the art. Step 940 may be imported to the computer. This can be done by the USB storage device or transferred electronically to the data collection 905 to computer or processor 840. In step 945 of data processing 905 processor 840 may parse CSV file for force values, height, and angle of rail 810 (As depicted in FIG. 8 and similar to other rails depicted in other Figures). The CSV file may also include a time stamp such that each force reading has a time stamp. In step 950 processor 840 may convert kilograms to newtons. In step 955 processor 840 may calculate normal force removing the effect of the lever. In step 960 processor 849 may add friction force and normal force together. In step 965 processor 840 may locate force data between limit switches and discard the rest of the data. In step 970 processor 840 may interpolate displacement over force. In step 975 processor 840 may fit the line to force to the displacement curve. In step 980 processor 840 may calculate the stiffness and put it in an array. Processor 840 may move to the next stop and move again to locate the force data between limit switches (825A-B) 965. Whether processor 840 moves on to the next step may be determined by whether processor 840 is receiving more information or if the input has ceased for a certain period of time processor may move to step 985 which may return the array of the stiffness values.

However, if the input is continuous processor may proceed to step 965. Force, time, and other sensor data are recorded in digital format synchronously. Thus, displacement and ultimately rigidity of the plant can be measured directly or inferred by a sensor using trigonometry based on the angle of rail 810.

Further, although specific implementations of the disclosure have been described and illustrated, the disclosure is not to be limited to the specific forms or arrangements of parts so described and illustrated. The scope of the disclosure is to be defined by the claims appended hereto, any future claims submitted here and in different applications, and their equivalents.

What is claimed is:

1. A rigidity measuring device comprising:
an arm comprising:
a rail comprising:
a limit switch disposed along the length of the rail; and
a base connected to a rail comprising:
a load cell attached to the base.

2. The rigidity measuring device of claim 1, further comprises:
a brace attached to one end of the base.

3. The rigidity measuring device of claim 2, further comprises:
a second load cell attached to the brace.

4. The rigidity measuring device of claim 3, further comprises:
a pivot joint that attaches to the second load cell on a first end and attaches to the rail on a second end.

5. The rigidity measuring device of claim 1, wherein the rail further comprises:
a second limit switch disposed along the length of the rail.

6. The rigidity measuring device of claim 1 further comprises a spring attaching to the rail and the base.

7. The rigidity measuring device of claim 1 further comprises:
a guard that extends over a portion of the rail and removably attaches to the base.

8. The rigidity measuring device of claim 1 further comprises:
one or more mounting pivots attached to the base.

9. The rigidity measuring device of claim 8 further comprises:
framing attached to the one or more mounting pivots wherein the framing attaches to a vehicle.

10. A rigidity measuring system comprising:
an arm comprising:
a rail comprising:
a limit switch disposed along the length of the rail;
a base connected to a rail comprising:
a load cell attached to the base;
a processor electronically connected to the limit switch and the load cell.

11. The rigidity measuring system of claim 10, wherein the arm further comprises:
a brace attached to one end of the base.

12. The rigidity measuring system of claim 11, wherein the arm further comprises:
a second load cell attached to the brace.

13. The rigidity measuring system of claim 12, wherein the arm further comprises:
a pivot joint that attaches to the second load cell at a first end and attaches to the rail at a second end.

14. The rigidity measuring system of claim 10, wherein the rail further comprises:
a second limit switch.

15. The rigidity measuring system of claim 10, wherein the arm further comprises:
a spring attaching to the rail and the base.

16. The rigidity measuring system of claim 10, wherein the arm further comprises:
a guard that extends over a portion of the rail and removable attaches to the base.

17. The rigidity measuring device of claim 10, wherein the arm further comprises:
one or more mounting pivots attached to the base.

18. The rigidity measuring system of claim 17 further comprises:
framing attached to the one or more mounting pivots wherein the framing attaches to a vehicle.

19. The rigidity measuring system of claim 10, wherein the processor collects and calculates data from both limit switches and both load cells.

20. The rigidity measuring system of claim 10, wherein the processor displays a plant rigidity result on a display screen.
